(19) United States
(12) Patent Application Publication
(10) Pub. No.: US 2025/0332185 A1
(43) Pub. Date: Oct. 30, 2025

(54) Use of a New Therapeutic Combination to Treat Chronic Venous Insufficiency

(71) Applicant: KÉRI PHARMA HUNGARY KFT., Debrecen (HU)

(72) Inventors: Judit Zsuga, Debrecen (HU); Levente Szendrei, Debrecen (HU)

(21) Appl. No.: 18/683,953

(22) PCT Filed: Aug. 18, 2022
(86) PCT No.: PCT/HU2022/000012

§ 371 Date: Feb. 15, 2024

(30) Foreign Application Priority Data
Aug. 19, 2021 (HU) P2100299

(51) Int. Cl. A61K 31/7048; A61K 31/4415; A61K 31/519; A61P 7/00

(52) U.S. Cl. CPC A61K 31/7048; A61K 31/4415; A61K 31/519; A61P 7/00

(57) ABSTRACT

The subject of the invention is a new medicinal combination. More specifically, the subject matter of the invention is a therapeutic combination for the treatment of venous insufficiency. More specifically, the subject of the invention is a combination of diosmin, folic acid and vitamin B6. It is also a subject matter of the invention to use the combination to prepare a pharmaceutical composition for the treatment of venous insufficiency and to use the combination or pharmaceutical composition according to the invention for the treatment of venous insufficiency. The results obtained with the compositions have shown that their use has contributed to a favourable therapeutic effect in all cases and that the reduction of homocysteine levels has been shown to have a positive effect on the cure of the disease even in the presence of normal or low homocysteine levels.

### FIELD OF THE INVENTION

The subject of the invention is a new medicinal combination. More specifically, the subject matter of the invention is a therapeutic combination for the treatment of venous insufficiency. More specifically, the subject of the invention is a combination of diosmin, folic acid and vitamin B6. It is also a subject matter of the invention to use the combination to prepare a pharmaceutical composition for the treatment of venous insufficiency and to use the combination or pharmaceutical composition according to the invention for the treatment of venous insufficiency.

### TECHNICAL BACKGROUND

Chronic venous disease is very common in Western societies and can occur in any pathology from telangiectasia (capillary stenosis), inflammation and thrombosis of the superficial veins (most commonly varicosities), to deep vein thrombosis. The forms of chronic venous insufficiency are:
functional: symptoms and complaints that occur with an intact structure
organic: pathologies caused by structural changes:
   diseases of the superficial venous system: varicosities, aneurysms
   diseases of the deep venous system: post-thrombotic syndrome, venous compression, venous malformations, Budd-Chiari syndrome, traumatic AV fistulas, deep venous aneurysms (Ministry of Health Professional Guideline, EÜM, 2013)

In Western Europe (France, Germany), varicosities affect 25-33% of women and 10-20% of men. The figures for Eastern Europe are less favourable. In Hungary, the prevalence of varicosities is close to 50%. It has been estimated that 1 in 100 people will develop a leg ulcer at some stage in their lives. Teleangiectasia and varicosities are seen in 12% of women aged 14-22 and in 41% of young adult women. Pregnancy increases the risk of developing chronic venous disease and exacerbates the symptoms of existing disease. The incidence is lower in men. The prevalence increases with age: more than 50% of the population over 65 years suffer from chronic venous disease. Women account for 73% of venous patients in this age group. Prolonged sedentary or standing work, sedentary lifestyle, poor diet, obesity, hormonal changes, as well as family history of venous disease can all be risk factors for the development of the disease.

The disease causes complaints that reduce the quality of life. It is characterised by aching pain, heavy leg sensation, limb fatigue, leg cramps (especially at night), itching, swelling, restless leg syndrome, cosmetic complaints. The complaints increase in the evening, when it is hot, after long periods of standing, sitting or travelling, and during menstruation.

Various diseases of the veins can occur at any age and in any part of the body (most commonly in the lower limbs), with a wide range of courses and outcomes. Underlying venous insufficiency are macrocirculatory and microcirculatory disorders. In macrocirculatory disorders, the main abnormality is the remodelling of venous walls and vein valve insufficiency, leading to increased venous pressure and lower limb oedema. Microcirculatory disorders are typically caused by biochemical changes as a result of disease-related inflammation.

In chronic venous disease, leukocyte infiltration has been detected in the vein walls and in the venous valves that provide venous circulation. Leukocytes associated with the endothelial cells lining the vein wall are activated and damage them by entering the vein wall. This results in a complex inflammatory process leading to valve damage, vein wall scarring and loss of elasticity. As a consequence of this process, in advanced stages, the leg muscle pump is unable to reduce the increased peripheral venous pressure during walking, the direction of venous circulation is reversed and a permanent, so-called “ambulatory venous hypertension” develops. This leads to abnormally high venous capillary pressures in the skin of the distal part of the leg, the most characteristic consequence of which is the formation of aqueous humour. This occurs at the lowest point of the body, around the ankle (where the pressure is greatest), as the increase in pressure causes the capillaries to dilate significantly and their permeability to increase. High molecular weight proteins (fibrinogen) leak into the tissues. The lymphatic circulation becomes overloaded, interstitial oedema and congestion develop. A further consequence of the increase in capillary permeability is that leukocytes adhere to the endothelial cells lining the capillaries and are activated there, causing further tissue damage. The latter result in the development of microcirculatory abnormalities.

Predisposing factors for the development of chronic venous insufficiency due to valve insufficiency include heredity, age (more common in older age), gender (varicosities are more common in women-male to female ratio: 1:4), race and geographical factors, pregnancy, and occupation involving standing or prolonged sedentary work.

### Treatment of Chronic Venous Insufficiency

Chronic venous insufficiency can be treated surgically or conservatively (non-surgically). The mainstay of conservative treatment is compression. Compression treatment with elastic stockings or elastic bandages is an essential method at all stages of chronic venous insufficiency, either alone or in combination with other treatment methods. However, the use of compression is a major source of discomfort for the patient and they often dismiss its use.

Another commonly used form of conservative treatment is the use of oral venoactive drugs. One indication for the use of oral venoactive drugs is to substitute compression (see below).

The medications used in chronic venous insufficiency are made from plants or synthetically. These medications fall into four groups: benzopyrones, saponins, other plant extracts and synthetic drugs. They act at two levels: on the one hand, in the macrocirculation, they induce changes in the vein walls that prevent the development of venous hypertension (pressure increase) and haemodynamic disturbances, and on the other hand, in the microcirculation, they inhibit the inflammation and the development of venous wall damage due to venous hypertension. Inhibition of venous hypertension is achieved by increasing venous tone. The increase in venous tone is achieved by most compositions (including diosmin, one of the components of the combination according to the present invention) via noradrenaline. These compositions inhibit the degradation of noradrenaline through inhibition of catechol-O-methyltransferase and thereby indirectly increase venous tone.

The drugs used today act on different elements of the inflammatory cascade, in particular inhibiting the leukocyte endothelial interaction. They also often have microcirculatory effects, such as increasing capillary resistance and reducing capillary permeability. The capillary protective effect of some compositions, such as diosmin, a component of the combination according to the present invention, is due in part to the inhibition of activation and adhesion of leukocytes to the endothelium. They improve lymphatic circulation, increase the number of lymph vessels and increase lymph flow. The formulations also have anti-inflammatory, free radical scavenging effects, inhibit the remodelling (i.e. scarring) of the connective tissue of the vein wall and reduce venous oedema.

### Indications for Oral Venoactive Drug Therapy:

For oedema and complaints at all stages of the disease.
In advanced stages, combined with compression, surgery, sclerotherapy.
For the treatment of leg ulcers, diosmin is used as a supplement to standard treatment.
Venoactive drugs can be used as a substitute for compression when contraindicated (arterial disease, skin infections, intolerance to stockings, lack of patient cooperation, warm environment).

One possible method of conservative treatment is described, for example, in European Patent No EP 2667904B1. This document describes the manufacture of compression stockings and tubes containing bioactive substances capable of promoting the beneficial adjuvant effects useful in the treatment of vascular diseases, notably venous insufficiency. In addition to the compression stockings and the novel knitted structure, the circular knitted tubes with a double-cylinder design presented in this document result in tubes which, thanks to their surface matrix, are able to release the active substances continuously during daily use of the product.

There are many products, but few proven effective treatments for venous disorders. Venoactive drugs were evaluated at the international consensus meeting in Sienna in 2005 and by various international vascular societies in 2008, according to the rules of evidence based medicine (EBM). Venoactive drugs were classified into 3 grades of recommendation (“A” being the highest level of recommendation, “C” the lowest level of recommendation). The use of diosmin, which is the basis of the present invention, has been assigned to the highest recommendation grade “A”.

The effects of diosmin are described in a number of literatures. For example, the Br. J. Surg. 2000, 87; 868-872, diosmin is reported to have anti-inflammatory effects, which play an important role in protecting blood vessels and contributing to the maintenance of proper blood circulation and venous tone. European Patent No EP 2531189B1 describes a combination formulation for the prevention and/or treatment of chronic venous insufficiency (CVI) comprising L-carnitine or propionyl-L-carnitine or its salt, troxerutin, diosmin and hesperidin.

### Therapeutic Practice

The division of venous diseases is based on the morphological and pathological dichotomy between the superficial and deep venous systems in the human body, and the acute and chronic diseases of both. On this basis, a distinction can be made between acute and chronic venous insufficiency, which can be caused by diseases of both the superficial and deep venous systems. Chronic venous insufficiency can be functional (symptoms and complaints with intact morphology) or organic (pathologies due to morphological changes). Among organic lesions, a distinction can be made between diseases of the superficial and deep venous system:
diseases of the superficial venous system
   primary varicosities:
   secondary varicosities,
   superficial truncal venous aneurysms
diseases of the deep venous system
   post-thrombotic syndrome
   vein compression,
   venous malformations,
   Budd-Chiari syndrome,
   traumatic AV fistulas,
   deep venous aneurysms.

In practice, depending on the type of underlying venous disease, but in general, all treatments are complemented by a so-called ‘base therapy’, which consists of compression and active exercise. In each case, the therapist adapts the therapy to be chosen to the morphological appearance of the lesions and the clinical staging of the disease, which reflects its progression.

Therapeutic options include complex conservative treatment, sclerotherapy, interventional radiological methods, local wound treatment and surgery. The combination of drugs of the present invention extends the therapeutic options of complex therapeutic treatment.

The groups of medications that can be used in complex conservative treatment are:
reducing vein wall permeability,
regulating coagulation conditions,
clot-dissolving,
platelet aggregation inhibitor,
oedema-relieving,
stimulating lymphatic circulation, and
preparations to improve microcirculation.

Therapeutic guidelines adapted to clinical staging recommend the use of complex conservative treatment at all clinical stages.

The generally accepted therapeutic guidelines in the profession, depending on the clinical stage (Widmer or CEAP classification), are:

### Widmer Classification

| Stage (symptoms) | Therapy |
|---|---|
| Stage I: | complex conservative treatment, |
| corona phlebectatica (abnormally | sclerotherapy (causing the vein |
| dilated veins around the ankle), | to scar) |
| subfascial congestion, oedema | |
| Stage II: | complex conservative treatment, |
| induration (hardening of the | interventional radiological |
| connective tissue), pigmentation | methods, |
| (here: dark discolouration of the | surgery. |
| skin along the course of a vein), | |
| congestive dermatitis (inflammation | |
| of the skin due to slowing of the | |
| circulation) | |
| Stage III: | surgery, |
| active or healed leg ulcers | local wound care, |
| | complex conservative therapy |

### CEAP Classification

| Stage | Symptoms | Therapy |
|---|---|---|
| 0 | no clinical signs | movement therapy, |
| | of venous disease | venotonics (vein constrictors), |
| | | compression (squeezing) |
| | | treatment |
| 1 | teleangiectasias (dilated | sclerotherapy (causing the |
| | capillaries) or reticular | vein to scar), |
| | veins | compression treatment |
| 2 | varicose (irregularly | sclerotherapy, surgery, |
| | dilated) veins | compression treatment |
| 3 | oedema | movement therapy, |
| | | venotonics, |
| | | compression treatment |
| 4 | skin lesions caused by venous | venotonics, |
| | disease pingmentation (dark | compression treatment, |
| | discolouration of the skin | dermatological treatment |
| | along the course of the vein), | surgery |
| | lipodermatosclerosis, venous | |
| | eczema | |
| 5 | the above skin lesions | venotonics, |
| | and healed leg ulcers | compression treatment, |
| | | dermatological treatment |
| | | surgery |
| 6 | the above skin lesions | surgery |
| | and active leg ulcers | venotonics, |
| | | compression treatment, |
| | | dermatological treatment |
| | | plastic surgery treatment |

At international level, there are similar general guidelines for the conservative management of chronic venous disease. The European Society for Vascular Surgery in its latest guideline for conservative therapy in 2015 distinguishes between two basic types of chronic venous disease:
1. chronic venous disease without ulceration (CEAP C0-C4, see below)
2. chronic venous disease with ulceration (CEAP C5-C6, see below).

The CEAP classification classifies chronic venous insufficiency according to four main aspects: clinical appearance, aetiology, anatomy and pathophysiology. Based on the clinical appearance, 7 grades of severity are distinguished:

### CEAP C0-C4:

C0: no visible or palpable signs of venous disease
C1: capillary dilatations (telangiectasias), reticular veins
C2: varicose veins
C3: oedema without skin lesions
C4: skin lesions of venous origin, dark skin discoloration (hyperpigmentation), venous eczema, lipodermatosclerosis

### CEAP C5-C6:

C5: skin lesions with healed ulcers
C6: active leg ulcer

The European Society of Vascular Surgeons recommends that the use of venotonics should be considered as a therapeutic alternative for the treatment of pain and oedema due to chronic venous disease in patients with stages C0-C4 (Wittens et al. Eur J Vasc Endovasc Surg (2015) 49, p. 702)

| Recommendation 34 | Class | Level | References |
|---|---|---|---|
| Venotonic drugs should be considered | IIa | A | 125, 275-282- |
| as a treatment option for swelling and | | | 285, 286 |
| pain caused by chronic venous disease. | | | |

According to the European Society of Vascular Surgeons recommendation (Wittens et al. Eur J Vasc Endovasc Surg (2015) 49, p. 702), the adjuvant use of sulodexide and micronized purified flavonoid fraction should be considered to complement compression therapy in patients with venous ulcer disease in stages C5-C6. The use of oral antibiotics, zinc, horse chestnut seed extract and pentoxifylline for the treatment of venous ulceration is not recommended according to the guideline.

| | Class | Level | References |
|---|---|---|---|
| Recommendation 35 | | | |
| Sulodexide and micronized purified | IIa | A | 295, 297- |
| flavonoid fraction should be considered | | | 299, 301 |
| as an adjuvant to compression therapy | | | |
| in patients with venous ulcers | | | |
| Recommendation 36 | | | |
| The routine use of zinc, oral | III | B | 208, 287-290, |
| antibiotics, horse chestnut seed | | | 294, 296 |
| extract and pentoxyfilline is not | | | |
| recommended in patients with venous | | | |
| leg uceration. | | | |

### Hyperhomocysteinemia and its Significance in Chronic Venous Disease

Homocysteine—HSCH₂CH₂CH(NH₂) COOH—is a sulphur-containing amino acid whose sole source is methionine. It plays a key role in the metabolism of two important amino acids, methionine and cysteine, in fact it links the metabolism of these two amino acids.

The amount of homocysteine in plasma is determined by the following factors:
synthesis from methionine,
catabolism (conversion to methionine and cysteine),
secretion from cells.

Several key enzymes of homocysteine metabolism require vitamins B6 and B12, and some products of folic acid metabolism play an important role in its conversion to methionine. (Kappelmayer and Muszbek 2021).

### Determination of Plasma Homocysteine Levels

Plasma homocysteine levels can vary depending on the method used for measurement. In general, 5-15 μmol/L is considered the reference range for fasting total homocysteine levels, but the risk threshold is within the reference range, around 12.5 μmol/L. Between 15 and 30 μmol/L is considered moderate, between 30 and 100 μmol/L moderate, and above 100 μmol/L severe hyperhomocysteinemia.

Homocysteine contains an active thiol group, which means that it is present in the plasma in a very low concentration in the free, reduced form, about 1%. The vast majority, about 70%, is attached to the cysteine side chains of proteins via a disulfide bridge. Most of the free (non-protein-bound) homocysteine—about 30% of total homocysteine—is bound to low molecular weight thiols. The level of total homocysteine in plasma is an important parameter for the development of various pathological conditions, in particular the predisposition to thrombosis, and is now almost exclusively used in laboratory diagnosis.

The analytical methods described for the determination of total homocysteine levels in plasma fall into two broad categories-enzymatic and chromatographic methods. The first step in all methods is the release of homocysteine bound to proteins or other thiols from disulfide bonds by reduction. In the immunological methods, the homocysteine is reduced and converted to S-adenosyl homocysteine (SAH) and an antibody against SAH is used in the immunological test. Among the chromatographic techniques, HPLC is the reference method for homocysteine determination where homocysteine is detected either fluorescently or electrochemically. (Kappelmayer and Muszbek 2021).

### Water-Soluble Vitamins Affecting Homocysteine Levels in Chronic Venous Ulcer Disease

The relationship between nutritional status and the pathological course of chronic wound healing is widely known, but most data come from studies of small patient populations with no control group. An exception is Renner et al. who compared the nutritional status of patients with chronic venous ulcers and their controls. A total of 50 people were included in the study and the participants' body mass index was determined, Mini-Nutritional Assessment (MNA) and Nutritional Risk Screening (NRS) questionnaires were taken and laboratory tests were performed. The 25 patients with chronic venous leg ulcers were compared with the 25 patients with acute surgical wounds. The patients in the “leg ulcer group” consisted of consecutive patients attending outpatient specialist clinics for chronic leg ulcers. The patients received optimal treatment for leg ulcers, consisting of appropriate modern wound management and compression therapy. Superinfection of the ulcer was excluded on the basis of the clinical appearance and laboratory tests. Leg ulcers persisted for at least 8 weeks in the patients in the “leg ulcer” group. The ulcer was characterised by laboratory tests and planimetric examination. 25 controls were included for surgery for skin cancer. Patients with chronic venous ulceration had higher body mass index, hyperhomocysteinemia and significantly lower levels of vitamins B6, B9, C and zinc. The authors concluded that patients with leg ulcers had a higher prevalence of obesity and qualitative malnutrition, i.e. inadequate intake of trace elements and vitamins, including lower levels of certain water-soluble vitamins such as vitamins B6, B9 and C (Renner, da Silva Garibaldi, Benson, Ronicke, & Erfurt-Berge, 2019).

### The Role of Homocysteine in Venous Circulatory Insufficiency

The role of homocysteine in increasing endothelial dysfunction, inflammation and vascular wall permeability has been previously suggested, effects that may potentially play a role in the development of complications of chronic venous disease.

The relationship between the severity of primary chronic venous disease and serum homocysteine levels was investigated in a cross-sectional study by Smith et al. (2016). A total of 282 patients with primary chronic venous disease were included in the study. The severity of venous disease was characterised by the CEAP staging. The results of the study showed that in this cohort study, homocysteine levels were higher in patients with more severe CEAP staging. The authors concluded that serum homocysteine levels are positively associated with the severity of primary chronic venous disease, suggesting that homocysteine may contribute to the development of venous disease complications. (Smith et al., 2016)

The association between homocysteine levels and chronic venous disease severity has also been investigated by meta-analysis. The most consistent finding was that homocysteine levels were elevated in patients with chronic venous disease and serum homocysteine concentrations were higher in patients with more advanced stage (Smith & Golledge, 2014). Of the three studies analysed to establish this relationship, the study by Sam et al. had the largest number of cases (Sam et al., 2003). By analysing data from their sample of 100 patients, the team found that hyperhomocysteinemia (>15 μmol/L) was significantly positively associated with clinical severity (p<0.001). Hyperhomocysteinemia was observed in 23% of patients with venous varicose veins, 20% of patients with edema, 39% of patients with skin lesions at the site of venous circulatory disturbance, 53% of patients with healed venous ulcers and 65% of patients with active venous ulcers.

Darvall et al. found a similar association, despite defining hyperhomocysteinemia with a higher threshold (>16.6 μmol/L). Patients with stasis dermatitis (CEAP C4) and active leg ulceration (CEAP C6) had significantly higher levels of homocysteine than the age- and gender-matched control sample. (Darvall et al., 2009)

In a similar study, Durmazlar et al. analysed homocysteine levels in patients with stasis dermatitis and leg ulcers. In total, stasis dermatitis was observed in 25 patients (17 males and 8 females; average age 36±5.97 years) and stasis ulcers were present in 40 patients (27 males, 13 females; average age 38.5±7.96 years). The control group consisted of 35 healthy individuals (25 men, 10 women; average age 36.9±6.49 years). The authors found that homocysteine levels were higher in patients with stasis dermatitis (p=0.00) and stasis ulcer (p=0.00) compared to healthy controls (median values of homocysteine levels (interquartile range) were 19.1 μmol/L (15-28), 18.98 μmol/L (15-29), and 8.1 μmol/L (5-12.2)). There was no significant difference in homocysteine levels between patients with stasis dermatitis and stasis ulcer (p=0.877). Kartal Durmazlar, Akgul, & Eskioglu, 2009, conclude that, given that hyperhomocysteinemia can be moderated by the administration of certain vitamins at normal vitamin levels, the development of leg ulceration may be prevented by reducing homocysteine levels at the stasis dermatitis stage.

A recent study demonstrated a strong, significant association between hyperhomocysteinemia and chronic venous ulcer disease. Reducing homocysteine levels with folate accelerated ulcer healing. The authors assumed that hyperhomocysteinemia is a marker of chronic venous ulcer disease and that treatments that reduce homocysteine levels promote ulcer healing (de Franciscis et al., 2015).

### Homocysteine, Vitamin B6 and Folic Acid Levels in Venous Ulcer Disease

The study by de Franciscis et al. (2015) analysed the prevalence of hyperhomocysteinemia in patients with venous ulcer disease and examined the effect of folic acid therapy on ulcer healing. Eighty-seven patients with venous ulcers were included in the study to determine the prevalence of hyperhomocysteinemia in this patient population. All patients received basic treatment for venous ulceration (surgical therapy #compression stockings). Patients in the hyperhomocysteinemia group received folic acid treatment in addition to the basic treatment (group A, 1.2 mg/day for 12 months), and patients without hyperhomocysteinemia (group B) received only the basic treatment. Ulcer healing was quantified with planimetric analysis. The prevalence of hyperhomocysteinemia among patients with venous ulcers included in the study was 62.06%. The cure rate was significantly higher in group A (78.75%) than in group B (63·33%) (P<0.05) (de Franciscis et al., 2015).

### Effects of Folic Acid and Vitamin B6 Supplementation on Plasma Homocysteine Levels in Healthy People

Dierkes et al. investigated the effect of folic acid supplementation (400 micrograms/day) on fasting homocysteine levels in healthy young women, compared with vitamin B6 (2 mg/day) or a combination of the two. Healthy young women with normal homocysteine levels were given folic acid alone, vitamin B6 alone or a combination of folic acid+vitamin B6 daily for four weeks. The combination reduced homocysteine levels by 17%, while supplementation with folic acid resulted in an 11.5% reduction. The effect of folic acid plus vitamin B6 was not significantly different from that of folic acid therapy alone. Vitamin B6 had no effect on plasma homocysteine levels. The authors conclude that homocysteine levels within the normal range can be reduced by supplementation with vitamins, including folic acid-containing vitamins. (Dierkers et al 1998)

### General Professional Understanding of the Management of Homocysteine Levels

Guidelines for the management of chronic venous insufficiency do not address the issue of how homocysteine levels are affected, i.e. current therapeutic practice does not consider homocysteine levels in the context of chronic venous insufficiency (Wittens et al. 2015). It is well known that the treatment of arterial and venous vascular impairment requires a completely different approach. However, from the point of view of the relationship between homocysteine and vascular disease, the professional judgement of the adverse effect on the arterial vasculature is relevant.

The adverse effects of homocysteine on the arterial vasculature have been addressed in previous professional guidelines. For example, the 2009 U.S. Preventive Services Task Force guideline discussed homocysteine levels as a non-traditional cardiovascular risk factor. The guideline found no convincing evidence that treating patients with high homocysteine levels improves cardiovascular outcomes. The guideline also mentions that the beneficial effect of reducing elevated homocysteine levels in primary prevention may be supported by the results of ongoing clinical trials (Calonge et al. 2009).

It is also noteworthy that the 2018 U.S. Preventive Services Task Force guideline no longer included elevated homocysteine levels among non-traditional risk factors, and only included ankle-brachial index, hsCRP level and CAC (a. coronaria Ca) score among non-traditional risk factors (Lin et al. 2018, Curry et al. 2018). It should also be noted that the 2019 guideline of the American Society of Cardiology does not discuss homocysteine levels as a relevant factor for primary prevention of cardiovascular disease (Arnett et al. 2019).

Similarly, the European Society of Cardiology guideline did not attribute any significance to homocysteine levels for cardiovascular risk (Piepoli et al. 2016). More specifically, this means that homocysteine levels are not relevant for medical decision-making and are considered irrelevant for therapy.

Therefore, it was not obvious to experts that lowering homocysteine levels could have any beneficial effect in venous insufficiency.

The general opinion of the profession that homocysteine levels do not need to be affected is well reflected in the PIL published in the American Society of Cardiology's journal Circulation.

The leaflet describes that elevated homocysteine levels are associated with an increased risk of venous thrombosis. It also describes that preparations (folic acid, vitamin B6, vitamin B12) are available to reduce elevated homocysteine levels. The leaflet says that homocysteine levels should be tested in patients younger than 20-30 years of age if the patient has a history of myocardial infarction, stroke, deep vein thrombosis, pulmonary embolism or homocystinuria that cannot be explained by other causes. In other patients, testing for homocysteine levels is not considered appropriate. The guidelines provide a negative answer to the question “If I have elevated homocysteine levels, do I need treatment?”, stating that “Although daily supplementation with folic acid, vitamin B6, or vitamin B12 is effective in reducing homocysteine levels, this reduction does not reduce the risk of cardiovascular disease, deep vein thrombosis, or pulmonary embolism (Moll et al. 2015).

The Cochrane Collaboration regularly conducts systematic reviews to examine the impact of homocysteine-lowering interventions on the risk of cardiovascular events, given that “An assumed risk factor for cardiovascular disease is elevated serum total homocysteine levels.” The systematic analysis was conducted in 2009, 2013, 2015 and 2017. The aim of the analysis is to determine whether homocysteine-lowering treatment is effective in preventing cardiovascular events, and whether it is effective in reducing all-cause mortality in patients with and without a history of cardiovascular disease. The analysis included randomised controlled trials with a follow-up of at least one year, where homocysteine-lowering therapy was used as an indication for the prevention of cardiovascular events. Compared with placebo, B6, B12 and folic acid therapy used as mono- or combination therapy showed no beneficial effect on myocardial infarction or death endpoints. In relation to acute stroke, little beneficial effect was found with enalapril+folic acid combination. This effect reflected the results of a large clinical trial involving 20 702 patients (Martí-Carvajal et al. 2017). A subgroup analysis of this study looked at data from 10 783 untreated hypertensive patients disaggregated by baseline homocysteine level (Qin et al. 2017). In this study, the antihypertensive effect of a daily combination of 10 mg enalapril+0.8 mg folic acid was compared with the effect of 10 mg enalapril monotherapy in untreated hypertensive patients. The study population was divided into three groups according to baseline homocysteine level: <10 μmol/L, 10-15 μmol/L, ≥15 μmol/L. After a (median) treatment period of 4.5 years, homocysteine levels decreased by 2.4 and 5.6 μmol/L, respectively, in the enalapril and enalapril+folic acid-treated groups with baseline homocysteine levels ≥15 μmol/L. Consistent with this, the effect on systolic blood pressure reduction was smaller in both treatment groups with baseline homocysteine levels ≥15 μmol/L (moderate reduction from ≈3 mm Hg to ≈1.4 mm Hg and from 1.70≈1.4 mm Hg to 1.17 mm Hg in the enalapril and enalapril+folic acid groups, respectively). A significant positive association was also observed between the reduction in homocysteine levels and the reduction in systolic blood pressure. There was no significant difference in the blood pressure lowering effect between the two treatment groups at the end of the 4.5-year treatment period.

At the end of the 4.5-year treatment period, homocysteine levels increased by 1.1 μmol/L in the enalapril group and remained unchanged in enalapril+folic acid-treated patients with baseline homocysteine levels between 10 and 15 μmol/L, and increased in both treatment groups in patients with baseline homocysteine levels <10 μmol/L (homocysteine levels increased by 1.7 μmol/L in the enalapril-treated group and by 1.3 μmol/L in the enalapril+folic acid-treated group). There was a positive association between the lowering effect of the treatments on homocysteine levels and the reduction in systolic blood pressure in the 4.5 years of enalapril and enalapril+folic acid treatment (P=0.040, n=10 783), but the association was weaker in the enalapril+folic acid treatment group (see Table). No significant effect was found for diastolic blood pressure.

TABLE 1
Reduction in systolic blood pressure after enalapril or enalapril +
folic acid treatment by category of baseline homocysteine levels

| | Reduction in systolic blood pressure | |
|---|---|---|
| Baseline | enalapril + folic acid | enalapril |
| homocysteine | n = 5 431 persons | n = 5 352 persons |
| <10 | ref. | ref. |
| 10-<15 | −0.67 (−1.42; 0.08) | −0.77 (−1.53; −0.01) |
| ≥15 | −1.17 (−2.13; −0.22) | −1.70 (−2.68; −0.72) |
| the trend p value | 0.02 | <0.001 |

The authors concluded that elevated homocysteine levels significantly reduced the antihypertensive effect of short- and long-term antihypertensive treatment with enalapril in previously untreated hypertensive patients (Qin et al. 2017). To the authors' knowledge, this study is the first to show that elevated homocysteine levels significantly reduce the efficacy of enalapril-based antihypertensive therapy in hypertensive patients. Considering that the pathomechanism of hypertension is different from that of chronic venous insufficiency and that this is a study result obtained under strictly controlled study conditions and is related to the interaction of homocysteine with antihypertensive agents, it can be concluded that the general professional consensus on homocysteine is that there is no justification for reducing homocysteine levels in either elevated or normal homocysteine levels.

However, the inventors of the invention realised that the specific regulation of homocysteine levels in chronic venous insufficiency could be crucial to curing the disease and reducing symptoms.

As discussed above, the guidelines for the management of chronic venous insufficiency do not address the issue of how homocysteine levels are affected, elevated homocysteine levels have previously been considered to be of relevance only in the context of cardiovascular disease, i.e. current therapeutic practice does not consider homocysteine levels in the context of chronic venous insufficiency. Although it has been established that homocysteine levels are higher in patients with venous insufficiency and there is evidence in the literature that lowering homocysteine levels may enhance symptom improvement in patients with venous disease, it is generally accepted and clinically practised that patients with normal homocysteine levels are not subject to diagnostic or therapeutic interventions by the treating physician related to homocysteine control.

In other words, the general professional consensus is that homocysteine levels in patients with chronic venous insufficiency are only a risk factor and that they only affect the patient's recovery if they are above normal levels.

In contrast, the inventors of the present invention have found that patients with chronic venous insufficiency can achieve significantly greater improvements in their condition when, after homocysteine levels have been reduced to normal, patients are given additional homocysteine-lowering agents in combination with oral venoactive drugs. In other words, the inventors have realised that the generally accepted normal homocysteine level in the treatment of venous insufficiency can be an obstacle to the rapid and effective treatment of the disease, and that reducing homocysteine levels below the normal range when administering oral venoactive drugs can significantly improve the treatment outcome.

In line with the above, the inventors of the present invention have developed a new therapeutic combination that can treat venous insufficiency much more effectively and quickly. Accordingly, the subject invention relates to a pharmaceutical combination for the treatment of venous insufficiency, comprising diosmin, folic acid and vitamin B6.

In using this combination, the inventors found that patients with normal homocysteinemia (normal homocysteine levels) had significantly greater symptomatic improvement and improved quality of life compared to the use of the oral venoactive agent alone, without homocysteine-lowering agents.

In accordance with the foregoing, it is also within the scope of the invention to use said combination according to the invention for the manufacture of a medicament for the treatment of venous insufficiency and to use said combination according to the invention for the treatment of venous insufficiency.

The combination according to the invention can be produced in a way well known to the person skilled in the art, for example by simply mixing the active ingredients. The combination may be administered in the form of a mixture, but also by simultaneous or sequential administration of the individual components. The amount of the combination to be administered and the dosage regimen are always determined by the treating physician, depending on the patient's age, physical condition, the severity of the symptoms and the condition to be treated.

The invention is illustrated by the following examples. Note that the embodiments described in the examples are purely illustrative and should in no way be considered as limiting our invention. It will be apparent to a person skilled in the art that obvious changes can be made to the embodiment of the invention as compared with the examples described in the following examples, which will also give the results described below, or lead to the conclusions described below. Such solutions all fall within the scope of the protection of our invention.

### EXAMPLES

The effects of diosmin, folic acid and vitamin B6 were examined. All three substances are commercially available and the active ingredients are 1000 mg diosmin, 3 mg folic acid and 20 mg vitamin B6. The diosmin plus combination means the simultaneous administration of each product in immediate succession. The order of administration of the products was arbitrary. It is obvious to a person skilled in the art that each of the preparations can be substituted by any equivalent preparation also commercially available.

### Example 1: Observation Test 1

The study analysed data from a total of 90 people retrospectively.

The analysis was based on consecutive patient data from outpatients who met the inclusion criteria. All patients had venous circulatory disorders, with severity of disease corresponding to CEAP stage 2 and CEAP stage 3. The male:female ratio in the population analysed was 1:2

In total, data from four different patient groups were examined:
Group 1: patients taking 1000 mg of diosmin daily (n=45)·
   30 subjects had homocysteine levels below the risk threshold (<12.5 μmol/L normal homocysteinemia group)·
   15 subjects had homocysteine levels above the risk threshold (>12.5 μmol/L hyperhomocysteinemia group)
Group 2: patients taking a daily combination of 1000 mg diosmin+6 mg folic acid+20 mg vitamin B6 (diosmin plus) (45 patients)·
   30 subjects had homocysteine levels below the risk threshold (<12.5 μmol/L normal homocysteinemia group)·
   15 subjects had homocysteine levels above the risk threshold (>12.5 μmol/L hyperhomocysteinemia group)

The observation period was 12 weeks, during which time patients took the prescribed medication continuously. Before starting the medication treatment, a physical examination (to determine the CEAP stage of chronic venous insufficiency) and homocysteine level determination were performed and a CIVIQ-20 questionnaire was filled out. At the end of the 12-week follow-up period, the tests were repeated. For continuous variables, normality testing was performed. Variables with a normal distribution (age, CIVIQ score at visits 1 and 2) were characterised by the mean+SD, while variables with a non-normal distribution (homocysteine level at visits 1 and 2) were characterised by the median (25; 75 percentile). Differences in the values of variables with normal distribution by subgroup was tested by t test, and difference in the values of variables with non-normal distribution by subgroup was tested by chi-squared test.

The factors determining the increase in CIVIQ score were analysed using simple and complex linear regression. In the composite model, age and gender were included as significant regressors. All factors (other than gender and age) that did not contribute significantly to the model were removed in each step of the modelling. The fit of the final composite linear model was characterised using the Ramsey RESET test and the Breusch-Pagan/Cook-Weisberg test. Model fit is indicated by a value of p>0.05. The composite model was also stratified according to baseline homocysteine level (normal vs. low), to investigate the interaction between baseline homocysteine level and the therapy used.

The baseline data and the results of Visit 2 at the end of 12 weeks of therapy are summarised in the table below:

| | diosmin treatment | | | |
|---|---|---|---|---|
| | normal | | diosmin plus treatment | |
| | homocysteine | hyperhomocysteine | normohomocystein = | hyperhomocysteine |
| Visit 1. | n = 30 persons | n = 15 persons | 30 persons | n = 15 persons |
| age (years) | 55.55 ± 8.91 | 59.48 ± 8.67 | 58.90 ± 7.56 | 60.78 ± 7.20 |
| gender | 10/20 | 5/10 | 10/20 | 5/10 |
| (male/female) | | | | |
| homocysteine | 8.00 (7.23; 8.83) | 14.74 (13.24; 15.79) | 7.89 (7.12; 8.69) | 15.04 (13.30; 16.37) |
| μmol/L | | | | |
| CIVIQ (score) | 66.09 ± 9.91 | 65.14 ± 8.45 | 66.70 ± 9.92 | 61.68 ± 8.76 |
| Visit 2. | n = 29 persons | n = 15 persons | n = 30 persons | n = 15 persons |
| homocysteine | 7.39 (7.65; 7.80) | 11.69 (11.52; 11.91) | 6.78 (6.45; 7.20) | 8.31 (7.98; 8.65) |
| μmol/L | | | | |
| CIVIQ (score) | 78.44 ± 8.75 | 79.71 ± 10.81 | 91.38 ± 8.90 | 89.12 ± 9.83 |
| CIVIQ change | −12.09 ± 1.23 | −14.56 ± 15.86 | −24.68 ± 14.71 | −27.44 ± 1.82 |
| (score) | | | | |

The following graphs show the effect of diosmin and diosmin plus preparations on CIVIQ score and serum homocysteine levels, broken down by baseline homocysteine level (normal vs. high).

It can be seen that diosmin plus induced a significantly greater increase in CIVIQ score after 12 weeks of treatment in both normal and high baseline homocysteine patients. It can also be seen that diosmin plus resulted in a significantly greater reduction in homocysteine levels after 12 weeks of treatment in both patients with normal and high baseline homocysteine levels, but the reduction was greatest in the group taking diosmin plus with high baseline levels.

### Simple Linear Regression-Determinants of Change in CIVIQ Score

(CIVIQ Visit 1 score-CIVIQ Visit 2 score)

| | | |
|---|---|---|
| | regression coefficient | |
| | (95% confidence | |
| Factor | interval) | p value |

| | | | |
|---|---|---|---|
| age (years) | 0.18 | (−0.14; 0.49) | 0.28 |
| gender (reference | −3.15 | (−8.68; 2.38) | 0.26 |
| category female) | | | |
| therapy (reference | −12.66 | (−17.19; −8.15) | <0.001 |
| category diosmin) | | | |
| homocysteine 1st | 0.052 | (−0.70; 0.81) | 0.89 |
| visit (μmol/L) | | | |
| CEAP (reference | −0.29 | (−5.71; 5.13) | 0.91 |
| category CEAP 2) | | | |
| homocysteine 2nd | 1.04 | (−0.42; 2.51) | 0.16 |
| visit (μmol/L) | | | |

### Multiple Linear Regression-Determinants of Change in CIVIQ Score-Full Sample

| | | |
|---|---|---|
| | regression coefficient | |
| | (95% confidence | |
| Factor | interval) | p value |

| | | | |
|---|---|---|---|
| age (years | 0.29 | (0.23; 0.57) | 0.034 |
| gender (reference | −3.16 | (−7.79; 1.54) | 0.186 |
| category female) | | | |
| therapy (reference | −13.42 | (−17.88; −0.96) | <0.001 |
| category diosmin) | | | |

Ramsey RESET test p=0.97
Breusch-Pagan/Cook-Weisberg test p=0.06

According to the multiple linear regression model, the most significant factor underlying the improvement in CIVIQ score (i.e., the increase in CIVIQ score at the 2nd visit) is the therapy used. For patients of the same age and gender, the use of the combination of diosmin plus results in a 13.42-point increase in the CIVIQ score.

### Multiple Linear Regression-Determinants of Change in CIVIQ Score in the Low (<12.5 μMol/L) Homocysteine Stratum

| | | |
|---|---|---|
| | regression coefficient | |
| | (95% confidence | |
| Factor | interval) | p value |

| | | | |
|---|---|---|---|
| age (years) | 0.071 | (−0.26; 0.41) | 0.67 |
| gender (reference | −5.10 | (−10.86; 0.66) | 0.08 |
| category female) | | | |
| therapy (reference | −12.87 | (−18.39; −7.35) | <0.001 |
| category diosmin) | | | |

### Multiple Linear Regression-Determinants of CIVIQ Score Variation in the High (≥12.5 μMol/L) Homocysteine Stratum

| | | |
|---|---|---|
| | regression coefficient | |
| | (95% confidence | |
| Factor | interval) | p value |

| | | | |
|---|---|---|---|
| age (years) | 0.85 | (0.38; 1.32) | 0.001 |
| gender (reference | −1.64 | (−9.25; 5.97) | 0.66 |
| category female) | | | |
| therapy (reference | −13.97 | (−21.08; −6.87) | <0.001 |
| category diosmin) | | | |

The results obtained by stratifying the composite model by homocysteine level at patient selection suggest the role of baseline homocysteine level in influencing the efficacy of the therapy used. It is observed that for patients with baseline homocysteine levels below the risk threshold, treatment of individuals of the same age and gender with 12 weeks of diosmin plus also results in a significant CIVIQ score increase of 12.87 points compared to diosmin treatment. A similar, albeit slightly larger, increase is also observed for those with elevated baseline homocysteine levels.

### 2. Example 2: Observation Test

Data from a total of 60 people were analysed retrospectively. Patients attending outpatient care consecutively who met the inclusion criteria were included in the retrospective analysis. Patients all had venous circulatory disorders with a severity of disease corresponding to CEAP stage 6. The male:female ratio in the analysed population was 1:2. The data of a total of four different patient groups were analysed as follows.
Group 1: patients taking 1000 mg of diosmin daily (n=30)
   15 patients had homocysteine levels below the risk threshold (<12.5 μmol/L, normal homocysteinemia group).
   15 patients had homocysteine levels above the risk threshold (>12.5 μmol/L hyperhomocysteinemia group)
Group 2: patients taking a daily combination of 1000 mg diosmin+3 mg folic acid+20 mg vitamin B6 (diosmin plus) (30 patients)
   15 patients had homocysteine levels below the risk threshold (<12.5 μmol/L normal homocysteinemia group).
   15 patients had homocysteine levels above the risk threshold (>12.5 μmol/L hyperhomocysteinemia group)
Group 3: patients taking a combination of 3 mg folic acid+20 mg vitamin B6 (diosmin plus) daily (30 patients)
   15 patients had homocysteine levels below the risk threshold (<12.5 μmol/L normal homocysteinemia group).
   15 patients had homocysteine levels above the risk threshold (>12.5 μmol/L hyperhomocysteinemia group)

The observation period was 12 weeks, during which time patients took the prescribed medication continuously. Before starting the medication treatment, a physical examination (to determine the CEAP stage of chronic venous insufficiency) and homocysteine level determination were performed. The area of the leg ulcer developed based on the venous circulatory disorder was also determined.

At the end of the 12-week observation period, the tests were repeated. For continuous variables, normality tests were performed. Variables with a normal distribution (age) were characterised by the mean+SD, variables with a non-normal distribution (homocysteine level at visits 1 and 2, leg ulcer area at visits 1 and 2) were characterised by the median (25; 75 percentile). For comparisons between two subgroups, the difference in the values of variables with normal distribution by subgroup was tested by t test, and the difference in the values of variables with non-normal distribution by subgroup was tested by chi-squared test.

The effectiveness of the therapy was characterised by the change in the area of the leg ulcer. Factors determining the change in ulcer size were analysed using simple and complex linear regression. In the composite model, age and gender were significant regressors. All factors (other than gender and age) that did not contribute significantly to the model were removed in each step of the modelling. The fit of the final composite linear model was characterised using the Ramsey RESET test and the Breusch-Pagan/Cook-Weisberg test. Model fit is indicated by a value of p>0.05. The composite model was also stratified according to baseline homocysteine level (normal vs. low), to investigate the interaction between baseline homocysteine level and the therapy used.

The baseline data and the results of Visit 2 at the end of 12 weeks of therapy are summarised in the table below:

| | diosmin treatment | | diosmin plus treatment | | Vitamin B6 + folic acid treatment | |
|---|---|---|---|---|---|---|
| | normal | | normal | | normal | |
| | homocysteine | hyperhomocysteine | homocysteine | hyperhomocysteine | homocysteine | hyperhomocysteine |
| Visit 1 | n = 15 persons | n = 15 persons | n = 15 persons | n = 15 persons | n = 15 persons | n = 15 persons |
| age | 67.12 ± 9.23 | 69.60 ± 10.93 | 71.58 ± 8.67 | 68.26 ± 7.96 | 65.92 ± 9.23 | 68.40 ± 10.93 |
| (years) | | | | | | |
| gender | 7/8 | 3/12 | 5/10 | 6/9 | 3/12 | 6/9 |
| (male/ | | | | | | |
| female) | | | | | | |
| homo- | 11.82 (11.59; 12.24) | 20.73 (20.16; 21.21) | 11.86 (11.36; 12.07) | 19.60 (19.38; 19.90) | 10.83 (9.77; 11.45) | 18.95 (18.65; 19.32) |
| cysteine | | | | | | |
| μmol/L | | | | | | |
| ulcer size | 8.11 (7.07; 8.73) | 13.62 (12.44; 13.85) | 8.95 (8.48; 9.79) | 12.28 (11.18; 12.96) | 11.93 (10.65; 12.72) | 13.07 (11.80; 13.32) |
| (cm²) | | | | | | |
| Visit 2 | n = 15 | n = 15 | n = 15 | n = 15 | n = 15 | n = 15 |
| homo- | 10.89 (10.21; 11.17) | 19.13 (18.75; 19.42) | 8.15 (7.57; 8.27) | 8.93 (8.53; 9.07) | 10.06 (9.08; 10.47) | 18.02 (17.62; 18.32) |
| cysteine | | | | | | |
| μmol/L | | | | | | |
| ulcer size | 7.09 (6.12; 7.67) | 10.56 (10.28; 10.91) | 4.02 (3.06; 4.42) | 5.58 (5.26; 5.82) | 9.61 (9.04; 10.64) | 11.96 (11.48; 12.38) |
| (cm²) | | | | | | |
| change in | 1.11 (0.64; 2.26) | 2.61 (2.01; 3.29) | 5.36 (4.51; 5.70) | 6.45 (5.74; 7.84) | 1.78 (0.72; 3.32) | 0.76 (−0.44; 2.02) |
| ulcer size | | | | | | |
| (cm²) | | | | | | |

### Simple Linear Regression-Determinants of Change in Leg Ulcer Area

(leg ulcer area 1st visit cm²-leg ulcer area 2nd visit cm²)

| | | |
|---|---|---|
| | regression coefficient | |
| | (95% confidence | |
| Factor | interval) | p value |

| | | | |
|---|---|---|---|
| age (years) | 0.44 | (−0.01; 0.10) | 0.12 |
| gender (reference | −0.44 | (−1.57; 0.68) | 0.44 |
| category female) | | | |
| therapy (reference | | | |
| category diosmin) | | | |
| diosmin plus | 4.14 | (3.38; 4.90) | <0.001 |
| vitamin B6 + folic acid | −0.38 | (−1.13; 0.39) | 0.34 |
| homocysteine 1st | 0.06 | (−0.06; 0.18) | 0.33 |
| visit (μmol/L) | | | |
| ulcer size 1st | 0.20 | (−0.05; 0.44) | 0.11 |
| visit (cm²) | | | |
| homocysteine 2nd | −0.28 | (−0.39; −0.18) | <0.001 |
| visit (μmol/L) | | | |
| Ulcer size at 2nd | −0.59 | (−0.72; −0.47) | <0.001 |
| visit (cm²) | | | |

### Multiple Linear Regression-Determinants of Change in Leg Ulcer Area-Full Sample

(leg ulcer area 1st visit cm²-leg ulcer area 2nd visit cm²)

| | | |
|---|---|---|
| | regression coefficient | |
| | (95% confidence | |
| Factor | interval) | p |

| | | | |
|---|---|---|---|
| age (years) | 0.022 | (−0.01; 0.05) | 0.18 |
| gender (reference | −0.69 | (−1.34; −0.48) | 0.04 |
| category female) | | | |
| therapy (reference | | | |
| category diosmin) | | | |
| diosmin plus | 4.13 | (3.38; 4.88) | <0.001 |
| vitamin B6 + folic acid | −0.37 | (−1.11; 0.38) | 0.33 |

Ramsey RESET test p=0.58
Breusch-Pagan/Cook-Weisberg test p=0.56

According to the multiple linear regression model, the most important factor behind the reduction in ulcer size is the therapy used. In patients of the same age and gender, the combination of diosmin plus is associated with a reduction in leg ulcer size of 4 13 cm².

### Multiple Linear Regression-Determinants of Change in Leg Ulcer Area—In the Low (<12.5 μMol/L) Homocysteine Stratum

(leg ulcer area 1st visit cm²-leg ulcer area 2nd visit cm²)

| | | |
|---|---|---|
| | regression coefficient | |
| | (95% confidence | |
| Factor | interval) | p |

| | | | |
|---|---|---|---|
| age (years) | 0.66 | (0.023; 0.11) | 0.003 |
| gender (reference | −0.15 | (−0.97; 0.67) | 0.71 |
| category female) | | | |
| therapy (reference | | | |
| category diosmin) | | | |
| diosmin plus | 3.78 | (2.82; 7.70) | <0.001 |
| Vitamin B6 + folic acid | 1.07 | (0.13; 2.02) | 0.03 |

### Multiple Linear Regression-Determinants of Change in Leg Ulcer Area—In the High (≥12.5 μMol/L) Homocysteine Stratum

(leg ulcer area 1st visit cm²-leg ulcer area 2nd visit cm²)

| | | |
|---|---|---|
| | regression coefficient | |
| | (95% confidence | |
| Factor | interval) | p |

| | | | |
|---|---|---|---|
| age (years) | −0.002 | (−0.04; 0.04). | 0.92 |
| gender (reference | −0.72 | (−1.56; 0.12) | 0.09 |
| category female) | | | |
| therapy (reference | | | |
| category diosmin) | | | |
| diosmin plus | 4.34 | (3.39; 5.29) | <0.001 |
| vitamin B6 + folic acid | −1.63 | (−2.58; −0.68) | 0.001 |

The results obtained with the stratification analysis of the composite model by homocysteine level at patient inclusion suggest the role of baseline homocysteine level in influencing the efficacy of the therapy used. It is observed that, in patients with baseline homocysteine levels below the risk threshold, treatment of individuals of the same age and gender with 12 weeks of diosmin plus also resulted in a significant reduction in leg ulcer size of 3.78 cm² greater than diosmin treatment. It should also be noted that treatment with B6+folic acid also produced a 1.07 cm² greater reduction than the reference diosmin.

The same trend was observed in patients with higher homocysteine levels at baseline, as treatment of individuals of the same age and gender with 12 weeks of diosmin plus resulted in a significant reduction in leg ulcer size of 4.34 cm², compared to the diosmin treatment. It is also an interesting observation that, among individuals with high baseline homocysteine, treatment with B6+folic acid was less effective than the reference diosmin treatment, as patients receiving vitamin B6+folic acid treatment had a 1.63 cm² smaller reduction in ulcer size than diosmin.

The above results show that a significantly better therapeutic effect was observed in patients treated with diosmin plus for chronic venous insufficiency of moderate severity (CEAP stage 2-3) and leg ulceration due to venous insufficiency. This beneficial effect resulted in a significant improvement in quality of life in both patient groups when diosmin plus was used compared to treatment with diosmin or vitamin B6 plus folic acid. A positive effect on the healing of leg ulcers was also observed in patients receiving diosmin plus treatment. The results of the inventors showed that, contrary to professional preconceptions, reducing homocysteine levels always contributes to a beneficial therapeutic effect, and of particular interest is that reducing homocysteine levels has a positive effect on disease cure even in normal or low homocysteine conditions.

1. A medicinal combination for the treatment of chronic venous insufficiency, containing a combination of diosmin, folic acid and vitamin B6.

2. The pharmaceutical combination according to claim 1, characterised in that the combination comprises diosmin, folic acid and vitamin B6.

3. The pharmaceutical combination according to claim 1, characterised in that the amount of diosmin is between 100 and 2000 mg, the amount of folic acid is between 1 and 10 mg and the amount of vitamin B6 is between 5 and 35 mg.

4. A therapeutic combination according to claim 1, characterised in that the amount of diosmin is between 500 and 1500 mg, the amount of folic acid is between 3 and 8 mg and the amount of vitamin B6 is between 15 and 25 mg.

5. A therapeutic combination according to claim 1, characterised in that the amount of diosmin is 1000 mg, the amount of folic acid is 6 mg and the amount of vitamin B6 is 20 mg.

6. A method of treating a patient with venous insufficiency, comprising administering the combination according to claim 1 to said patient.

7. The method of claim 6, wherein the patient with venous insufficiency has a high serum homocysteine level.

8. The method of claim 6, wherein the patient with venous insufficiency has a normal serum homocysteine level.

9. The method of claim 6, wherein the patient with venous insufficiency has a low serum homocysteine level.

10. (canceled)

11. A pharmaceutical composition for the treatment of chronic venous insufficiency, said pharmaceutical composition comprising a therapeutic combination according to claim 1.

12. A method of treating a patient with venous insufficiency comprising administering the pharmaceutical composition according to claim 11 to said patient.

13. The method of claim 12, wherein the patient with venous insufficiency has a high serum homocysteine level.

14. The method of claim 12, where the patient with venous insufficiency has a normal serum homocysteine level.

15. The method of claim 12, wherein the patient with venous insufficiency has a low serum homocysteine level.
